(19) United States
(12) Patent Application Publication
(10) Pub. No.: US 2021/0204835 A1
(43) Pub. Date: Jul. 8, 2021

(54) Testicular Volume Measuring Device

(71) Applicants: Steve Ramelot, Huntington Beach, CA (US); Peter Bradshaw, Westminster, CA (US)

(72) Inventors: Steve Ramelot, Huntington Beach, CA (US); Peter Bradshaw, Westminster, CA (US)

(21) Appl. No.: 17/141,023

(22) Filed: Jan. 4, 2021

Related U.S. Application Data
(60) Provisional application No. 62/956,458, filed on Jan. 2, 2020.

(51) Int. Cl. A61B 5/107; A61B 5/00; A61B 50/30

(52) U.S. Cl. CPC A61B 5/1073; A61B 2050/0066; A61B 50/30; A61B 5/4387

(57) ABSTRACT

A testicular volume measuring device has a container, a lid, a sealing ring, and a plurality of indicia. The lid is removably attached atop the container, and the plurality of indicia is positioned on a lateral wall of the container, which is constructed of a transparent material and delineates an internal cavity. The lid has multiple lid members, which are removably attached to each other. The lid members have cutouts which form a central aperture on the lid, within which the sealing ring is secured during use. One or more testicles may be placed through the sealing ring, around which the lid members are affixed, and the lid is attached atop the container. Fluid volume measurements using the indicia before and after placement of the testicles into a quantity of fluid contained within the cavity enable measurement of the volume of the testicles through fluid displacement.

The current application claims a priority to the U.S. Provisional Patent application Ser. No. 62/956,458 filed on Jan. 2, 2020. The current application is filed on Jan. 4, 2021, whereas Jan. 2, 2021 and Jan. 3, 2021 were on a weekend.

### FIELD OF THE INVENTION

The present invention relates generally to bodily measuring devices. More particularly, the present invention relates to testicular volume measuring devices.

### BACKGROUND OF THE INVENTION

The size of one's testicles is an important indicator of one's virility and can help determine the health of one's sperm. Practices such as hormone replacement therapy, lifestyle, and natural supplementation can affect and change the size of one's testicles. Hormone replacement therapy can change the size of the user's testicles, so the size of the user's testicles will need to be constantly measured and tracked to ensure the treatment is progressing safely as prescribed. Natural supplementation and lifestyle changes can also affect the size of the user's testicles, the results of which need to be measured and tracked for effectiveness.

Current devices for measuring testicles do not efficiently track the size of the user's testicles. Calipers are a known measuring device for testicles. However, calipers are both uncomfortable and only measure the girth of the user's testicles, not the volume. Other measuring tools such as orchidometers can only give rough estimates of the user's testicle size and not precise measurements. The best tool for measuring the user's testicle size is an ultrasound. However, it would be inefficient to use an ultrasound to track the size of one′ testicles on a daily basis. Therefore, there is a need for a device that can be used to precisely measure the size of the user's testicles on a daily basis.

The present invention is a device for measuring the volume of the user's testicles. The present invention allows one to precisely measure the size of one's testicles on a daily basis. The present invention does not require assistance from a doctor to be used. The present invention is comfortable to use and measures the size of the user's testicles based on the amount of fluid displaced by the user's testicles within the device.

Additional advantages of the invention will be set forth in part in the description which follows, and in part will be obvious from the description, or may be learned by practice of the invention. Additional advantages of the invention may be realized and attained by means of the instrumentalities and combinations particularly pointed out in the detailed description of the invention section. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

FIG. 1 is a front view of one embodiment of the present invention.

FIG. 2 is an elevated perspective view of one embodiment of the present invention with the lid detached from the container.

FIG. 3 is an elevated perspective exploded view of one embodiment of the present invention.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

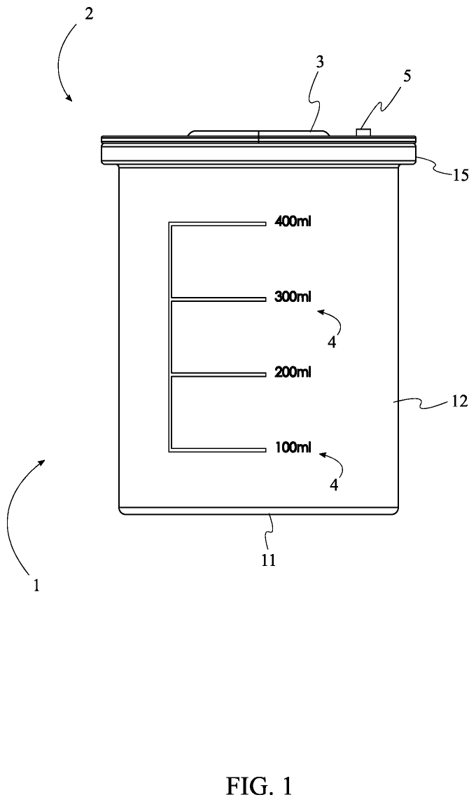

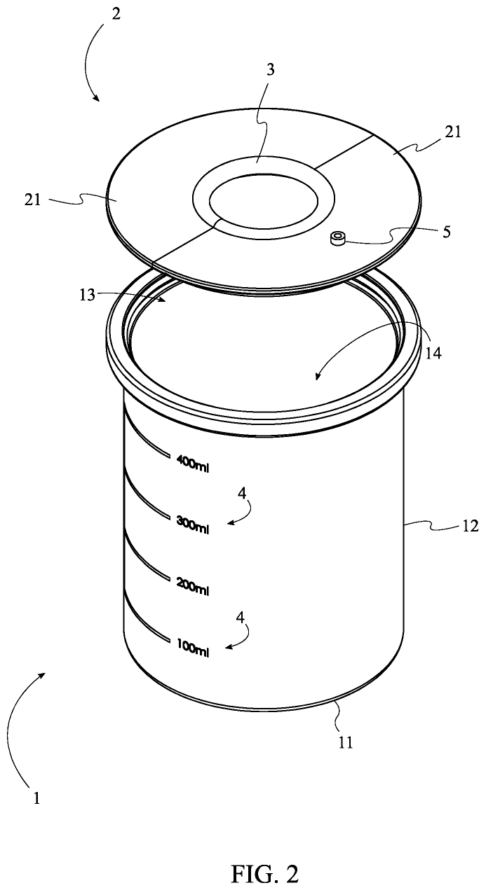

The present invention is a measuring device for measuring the volume of at least one human testicle. The present invention may be used to obtain precise measurements for the volume of at least one testicle by measuring the amount of fluid displaced by the testicle(s). Generally, referring to FIGS. 1-2, the preferred embodiment of the present invention comprises a container 1, a lid 2, a sealing ring 3, and a plurality of indicia 4. The lid 2 is removably attached atop the container 1, and the plurality of indicia 4 is positioned laterally on the container 1. The lid 2 comprises a plurality of lid members 21, the plurality of lid members 21 being removably attached to each other around the sealing ring 3, wherein the sealing ring 3 is configured to receive at least one human testicle therethrough into the container 1.

It should be understood that the plurality of indicia 4 as referred to herein comprises a series of individual indicia markings arranged and configured to correspond by height on the container to a plurality of volumes of liquid contained therein. For example, as illustrated in FIG. 1, if the container 1 has a diameter of ten centimeters, the plurality of indicia 4 of the present invention may comprise a first indicium marking of “100 ml” at a height of h=100/(π*5*5)=1.27 centimeters from a base 11 of the container 1, a second indicium marking of “200 ml” at a height of 2.55 cm, a third indicium marking of “300 ml” at a height of 3.82 centimeters, and so on. Of course, the particular scale, arrangement of sub-indicium, and other attributes of the plurality of indicia 4 may vary in different embodiments as desired and may utilize either metric or Imperial units as desired.

The container 1 of the present invention serves to hold a quantity of fluid, such as, but not limited to water, used to measure the volume of at least one testicle. By adding a quantity of fluid with a known initial volume to the container 1 (or adding a quantity of fluid and measuring its initial volume with the plurality of indicia 4), placing a testicle through the sealing ring 3 and the lid 2 into the quantity of fluid, observing a final volume measured by the plurality of indicia 4, and subtracting the final volume from the initial volume, the volume of a testicle or testicles may be measured using the present invention. In the preferred embodiment of the present invention, the container 1 is constructed from a translucent or transparent material, so that the current volume of liquid within the container 1 may be ascertained by observing the current height of liquid through the translucent or transparent material of the container 1 and matching said height to the closest volume marking on the plurality of indicia 4.

More particularly, the container 1 comprises a base 11, a lateral wall 12, an opening 13, and a cavity 14. The lateral wall 12 is connected atop the base 11, and is preferably cylindrical in geometry, though it should be understood that the container 1 of the present invention should not be necessarily limited to any particular geometry, so long as the container 1 is able to adequately hold a fluid in a manner suitable for measuring with the plurality of indicia 4 in accordance with the spirit and purpose of the present invention. For example, in some embodiments, the container 1 may have rectilinear geometry. Alternatively, in some embodiments, the container 1 may have oblong geometry in order to conform more closely with the general shape of a human testicle. So long as the base 11 functions to retain fluid within the container 1, the base 11 may take any desirable form, and is generally considered to have corresponding geometry to the lateral wall 12. In various embodiments, the plurality of indicia 4 may be positioned externally or internally on the lateral wall 12 of the container 1 as desired in order to properly perform its measuring function.

The cavity 14 is delineated within the container 1 by the base 11 and the lateral wall 12, while the opening 13 is positioned opposite the base 11 along the lateral wall 12, such that the opening 13 is positioned adjacent to and traverses into the cavity 14.

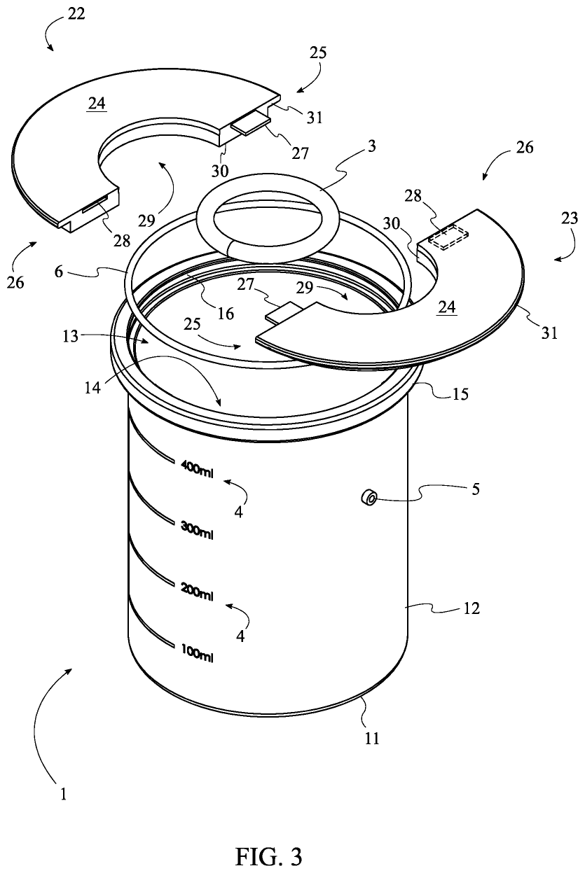

Some embodiments of the present invention may further comprise a check valve 5, which is configured to unidirectionally transfer fluid into the cavity 14 of the container 1. The volume of fluid entering the container 1 may be predetermined and envelop at least one testicle positioned therein. In some embodiments, the check valve 5 may be configured to receive fluid delivered through a syringe, wherein the syringe is utilized to predetermine the volume of fluid entering the container 1 through the check valve 5. The exact location of the check valve 5 is not of great importance and may vary in different embodiments. In some embodiments, the check valve 5 is positioned on and traverses through the lid 2, more particularly one of the plurality of lid members 21 as shown in FIGS. 1-2. In some embodiments, the check valve 5 is positioned on and traverses through the lateral wall 12 of the container 1 as shown in FIG. 3.

As previously mentioned, the lid 2 is removably attached atop the container 1. More specifically, the lid 2 is removably attached adjacent to the opening 13. While it is contemplated that in various alternative embodiments, the plurality of lid members 21 may comprise any desirable number of lid members in any desirable configuration pursuant to the spirit of the present invention, in the preferred embodiment, the plurality of lid members 21 comprises a first lid member 22 and a second lid member 23, as shown in FIG. 3. The first lid member 22 and the second lid member 23 are removably attached to each other and the sealing ring 3 is removably and concentrically positioned between the first lid member 22 and the second lid member 23.

More specifically, in the preferred embodiment, the first lid member 22 and the second lid member 23 each comprise a body 24, a first end 25, a second end 26, a first attachment portion 27, a second attachment portion 28, and a semicircular cutout 29. The body 24 preferably arcuately traverses between the first end 25 and the second end 26, such that the body 24 of each of the first lid member 22 and the second lid member 23 is a semi-circular arc of constant thickness. However, this is not a strict requirement, and the body 24 may have other geometries in alternative embodiments.

For each of the first lid member 22 and the second lid member 23, the first attachment portion 27 is positioned at the first end 25 of the body 24, and the second attachment portion 28 is positioned at the second end 26 of the body 24. The semicircular cutout 29 is concentrically positioned with the body 24 between the first end 25 and the second end 26. The first attachment portion 27 of the first lid member 22 and the second attachment portion 28 of the second lid member 23 are removably attached to each other. Similarly, the second attachment portion 28 of the first lid member 22 and the first attachment portion 27 of the second lid member 23 are removably attached to each other. The sealing ring 3 is positioned concentrically within the semicircular cutouts 29 of both the first lid member 22 and the second lid member 23, when the first lid member 22 and the second lid member 23 are attached to each other. Accordingly, the lid 2 assembly can receive a testicle such that the scrotum prevents fluid from exiting the container 1 through the sealing ring 3. The lid 2 assembly can create a seal around the scrotum and on the container 1 to prevent fluid from exiting the container 1 while at least one testicle is being measured.

Thus, in the preferred embodiment of the present invention, the first lid member 22 and the second lid member 23 are mirror images of each other (more specifically, doubly mirrored, both in X and Y directions, wherein the lid 2 is substantially flat in an X-Y plane), with the exception of the check valve 5 being present on one of the lid 2 members in some embodiments; accordingly, when the first lid member 22 and the second lid member 23 are joined together by their respective attachment portions, the bodies of the lid members form a circular arc of constant thickness, while the semicircular cutouts 29 of the lid members form a circular aperture traversing concentrically through the lid 2 within which the sealing ring 3 is secured when in use.

Moreover, in the preferred embodiment of the present invention, the first attachment portion 27 is a protrusion for each of the first lid member 22 and the second lid member 23, while the second attachment portion 28 is a receptacle for each of the first lid member 22 and the second lid member 23. The protrusion is connected to the first end 25, while the receptacle traverses into the second end 26, for each of the first lid member 22 and the second lid member 23. The protrusion of the first lid member 22 is removably positioned, preferably with a press fit connection, within the receptacle of the second lid member 23, while the protrusion of the second lid member 23 is similarly removably positioned within the receptacle of the first lid member 22 in order to removably attach the first lid member 22 and the second lid member 23 together.

It should be noted that although a specific means of attachment between the first lid member 22 and the second lid member 23 has been specified in the preceding disclosure, the present invention should not be considered as limited to said means, and any other suitable attachment means may be implemented as desired in different embodiments. For example, in some embodiments, the first attachment portion 27 and the second attachment portion 28 of each of the first lid member 22 and the second lid member 23 may be, for example, magnets, hook and loop tape, clasps, snap fasteners, or any other suitable fasteners or attachment hardware or other attachment means.

In some embodiments of the present invention, an O-ring 6 is further comprised. Moreover, the first lid member 22 and the second lid member 23 each comprise a lower body portion 30 and an upper flange portion 31, while the container 1 further comprises a rim 15 and an annular groove 16. The rim 15 is perimetrically connected to the lateral wall 12 of the container 1 adjacent to the opening 13, opposite the base 11, while the annular groove 16 laterally traverses into an interior side of the rim 15 adjacent to the opening 13. The O-ring 6 is positioned within the annular groove 16. The lower body portion 30 of each of the first lid member 22 and the second lid member 23 are positioned within the rim 15 when the lid 2 is removably attached atop the container 1, wherein the O-ring 6 is abutted against the lower body portion 30 when the lid 2 is removably attached atop the container 1, thus forming a continuous seal around the opening 13 of the container 1 against any liquid escaping during use. Further, the upper flange portion 31 of each of the first lid member 22 and the second lid member 23 is positioned atop the rim 15 when the lid 2 is removably attached atop the container 1 to the rim 15. Alternatively to embodiments comprising the O-ring 6, in some embodiments, a proper seal between the lid 2 and the container 1 is accomplished through a compression fit, and in some embodiments, the compression fit incorporates tapered walls of the lid 2 and container 1, or rim 15 of the container 1, as is appropriate in various embodiments.

Although the invention has been explained in relation to its preferred embodiment, it is to be understood that many other possible modifications and variations can be made without departing from the spirit and scope of the invention as hereinafter claimed.

What is claimed is:

1. A testicular volume measuring device comprising:
a container;
a lid;
a sealing ring;
a plurality of indicia;
the lid being removably attached atop the container;
the plurality of indicia being positioned on the container;
the lid comprising a plurality of lid members; and
the plurality of lid members being removably attached to each other around the sealing ring, wherein the sealing ring is configured to receive at least one human testicle therethrough into the container.

2. The testicular volume measuring device as claimed in claim 1, wherein the container is constructed from a translucent material.

3. The testicular volume measuring device as claimed in claim 1 comprising:
the container comprising a base, a lateral wall, an opening, and a cavity;
the lateral wall being connected atop the base;
the cavity being delineated within the container by the base and the lateral wall;
the opening being positioned opposite the base along the lateral wall, wherein the opening traverses into the cavity; and
the plurality of indicia being positioned on the lateral wall.

4. The testicular volume measuring device as claimed in claim 3 comprising:
the lid being removably attached adjacent to the opening.

5. The testicular volume measuring device as claimed in claim 1 comprising:
a check valve; and
the check valve traversing through the lid, wherein the check valve is configured to unidirectionally transfer fluid into the container.

6. The testicular volume measuring device as claimed in claim 1 comprising:
a check valve; and
the check valve traversing through a lateral wall of the container, wherein the check valve is configured to unidirectionally transfer fluid into the container.

7. The testicular volume measuring device as claimed in claim 1 comprising:
the plurality of lid members comprising a first lid member and a second lid member;
the first lid member and the second lid member being removably attached adjacent to each other; and
the sealing ring being removably and concentrically positioned between the first lid member and the second lid member.

8. The testicular volume measuring device as claimed in claim 1 comprising:
the plurality of lid members comprising a first lid member and a second lid member;
the first lid member and the second lid member each comprising a body, a first end, a second end, a first attachment portion, a second attachment portion, and a semicircular cutout;
the body arcuately traversing between the first end and a second end;
the first attachment portion being positioned at the first end;
the second attachment portion being positioned at the second end;
the semicircular cutout being concentrically positioned with the body between the first end and the second end;
the first attachment portion of the first lid member and the second attachment portion of the second lid member being removably attached to each other;
the second attachment portion of the first lid member and the first attachment portion of the second lid member being removably attached to each other; and
the sealing ring being concentrically positioned within the semicircular cutouts of the first lid member and the second lid member, when the first lid member and the second lid member are attached to each other.

9. The testicular volume measuring device as claimed in claim 8, wherein the body of each of the first lid member and the second lid member is a semi-circular arc of constant thickness.

10. The testicular volume measuring device as claimed in claim 8 comprising:
the first attachment portion being a protrusion for each of the first lid member and the second lid member;
the second attachment portion being a receptacle for each of the first lid member and the second lid member;
the protrusion being connected to the first end;
the receptacle traversing into the second end;
the protrusion of the first lid member being removably positioned within the receptacle of the second lid member; and
the protrusion of the second lid member being removably positioned within the receptacle of the first lid member.

11. The testicular volume measuring device as claimed in claim 1 comprising:
an O-ring;
a first lid member and a second lid member of the lid each comprising a lower body portion and an upper flange portion;
the container further comprising a rim and an annular groove;
the rim being perimetrically connected to the lateral wall adjacent to the opening, opposite the base;
the annular groove laterally traversing into the rim adjacent to the opening;
the O-ring being positioned within the annular groove;
the lower body portion of each of the first lid member and the second lid member being positioned within the rim when the lid is removably attached atop the container, wherein the O-ring is abutted against the lower body portion when the lid is removably attached atop the container; and
the upper flange portion of each of the first lid member and the second lid member being positioned atop the rim when the lid is removably attached atop the container.
